(19) United States
(12) Patent Application Publication
(10) Pub. No.: US 2006/0030700 A1
(43) Pub. Date: Feb. 9, 2006

(54) D-ribose, its monohydrate and their caramels

(76) Inventor: Keith Earl Kenyon, Sherman Oaks, CA (US)

Correspondence Address: KEITH E. KENYON, M.D., 4501 NAGLE AVENUE, SHERMAN OAKS, CA, 91423, US

(21) Appl. No.: 11/236,812

(22) Filed: Sep. 26, 2005

Related U.S. Application Data
(63) Continuation of application No. 10/886,070, filed on Jul. 7, 2004.

(51) Int. Cl. C07H 3/00

(52) U.S. Cl. 536/124

(57) ABSTRACT

This invention discloses the formation of a new molecule when one mole only of D-ribose crystalline powder is mixed with one mole only of water at room temperature which new substance is entirely different from the precursor molecule, and a monohydrate of D-ribose is the likely newly discovered molecule formed with both the precursor and the result able to form caramels at below 100° C. so can form relatively low temperature caramel taste when mixed with other sugars.

### RELATED APPLICATIONS

This patent application is a continuation in part of patent application Ser. No. 10/886,070. It is also related to patent application Ser. No. 10/913,181 and provisional patent application No. 60/620,115.

### FIELD OF THE INVENTION

This invention is in the field of organic chemistry and more particularly in the field of carbohydrate chemistry.

### BACKGROUND OF THE INVENTION

D-ribose is a synthesized sugar now being marketed to consumers without identifying its true nature, having unique characteristics not before understood nor disclosed accurately to the United States Food and Drug

Administration (FDA). It does not exist in the free-state in nature as do all other commercial, consumable dextrorotary sugars. While any sugar can be synthesized, including with various catalytic means from formaldehyde groups, and D-ribose can also, all other natural sugars that are used for human consumption exist at least in part in a free-state such as sucrose does in sugar cane, glucose does in grapes, fructose does in citrus fruits, and xylitol does in birch. Of course, within any given fruit or plant more than one such free sugar can exist simultaneously.

On the other hand, D-ribose does not, only in combination with one or more other radicals. For example, it is made from D-glucose in the pentose phosphate pathway in the mitochondria by glucose in its free-state being phosphorylated first and the phosphorylated glucose then having a carbon atom removed producing phosphorylated D-ribose. No one would call glucose-6-phosphate a free form of glucose but rather a compound. The same must be attributed to ribose, but the CEO of the leading company marketing ribose, Bioenergy, Inc., wrote on Aug. 23, 2004 that D-ribose “is present in its free (my italics) form as Ribose-5-Phosphate”. In fact free D-ribose plays no role in the living body of both man and animals because in such an environment it is extremely unstable and instantaneously loses its free-state, which is the main reason for this disclosure. As a consequence, crystalline D-ribose must be harvested from one or more of its compounds in living material, today preferably by various forms of Bacillus subtilis. U.S. Pat. No. 3,970,522 was the first patent filed in the biosynthesis of D-ribose from B. subtilis for the purpose of the further synthesis of riboflavin. At no time does D-ribose become free until crystallization occurs as can be ascertained by the following excerpt from that disclosure:

“The cultivation is conducted aerobically, for example by shaking culture or submerged culture under sparging and stirring. The incubation temperature is usually selected from within the range of 20 to 45 degrees C., depending upon the temperature suited for the particular organism to grow and accumulate D-ribose. The pH of the medium is preferably somewhere between about 5 and about 9. To maintain the pH within the optimum range throughout the cultivation period, one may incorporate from time to time such a neutralizer as hydrochloric acid, sulfuric acid, aqueous ammonia, ammonia gas, an aqueous solution of sodium hydroxide, calcium carbonate, slaked lime, etc. Ordinarily, a substantial amount of D-ribose accumulates in the medium in about 2 to 5 days. The D-ribose thus accumulated can be easily recovered, for example, by the following procedure. Namely, the culture broth is first filtered or centrifuged, whereby the cells can be removed with great ease. Then, the filtrate is desalted and decolorized by treatment with activated carbon and ion exchange resin and, then, concentrated. To the concentrate is added an organic solvent such as ethanol, whereupon D-ribose crystals separate. Whether the above or other method is employed, D-ribose can be easily recovered.”

When comparing D-ribose to the pentose polyol, xylitol, both have an endothermic property, but xylitol's is from a net negative heat of solution and ribose's is not. Both sugars can be produced by biosynthesis from specified microbes that tend to accumulate the respective sugars, then using desalting means as described above, but after this they depart because xylitol also exists in a free-state in fruits, vegetables and as a wood sugar in birch, while D-ribose does not exist in a free-state in the natural environment so always requires synthesis one way or another to produce the free, non-combined sugar to exhibit the endothermic sensation at room temperature. Some of the best procedures for the laboratory preparation of ribose involve the hydrolysis of yeast nucleic acid which existed prior to utilization of B. subtilis, the latter made preferably today as fed-batch production from glucose and xylose mixtures, which have the least front-end expense.

Once synthesized, crystalline D-ribose has a high capability of reacting with the carboxylic acid moiety, not only with amino acids, slowly at room temperature and rapidly at higher temperatures, but also with the sodium salt of pyrrolidone carboxylic acid (NaPCA) at room temperature. It also caramelizes (oxidizes) readily at relatively low temperatures but such temperatures are much higher than room and body temperatures. On the other hand, xylitol has a very low level of reactivity this way in contrast to D-ribose's high Maillard and caramelizing reactivity at temperatures far lower than other sugars. Therefore, D-ribose is unique amongst those sugars which are consumable by man and other mammals, including domesticated pets, in that it only exists in nature as a radical, and its radical attachments must be forcibly removed chemically to produce the free-state in every case.

Care must be taken to understand what crystalline D-ribose actually is compared to all other crystalline sugars, always a synthetic sugar that is not natural in the free-state. Thus in 1933 when Levene and Stiller reacted D-ribose with acetone, the D-ribose they used had to be laboratory-grade which had to be synthesized to begin with. This sometimes is confusing even to USPTO patent examiners who have gone so far as to cite the hexose hamamelose (alpha-oxymethyl-ribose), a Witch Hazel wood sugar that exists in the free-state, as being the same chemically as crystalline D-ribose because the two can react with the same chemical such as NaPCA. Such a narrow point of view is obviously incorrect for a number of reasons, including the fact that crystalline D-ribose is not a natural substance that can also be biosynthesized as is the case with hamamelose but is a synthesized reactant only, whether the reaction takes place in the laboratory or in a human body. Because of its unique unstable nature D-ribose must react in some manner with considerable other molecules or radicals and even spontaneously at room temperature when coming in contact with at least one and perhaps more of such molecules or radicals.

The question arises that in the pentose phosphate pathway's numerous reactions over its 72-hour span, endothermic, exothermic and condensation reactions all occur, so could all of these be manifestations of the exact same reaction? The answer is no. Since endothermic, exothermic or thermoneutral reactions for that matter cannot be interchanged with respect to enthalpy, they therefore can only be one at a time, whatever one it is. Condensation reactions can be part of all three but only one at a time. Therefore, if D-ribose were to be reacted with acetone, a condensation reaction could follow in which monoacetone-d-ribose is the result with the separation out of one free water molecule. Since a new larger molecule plus water is formed from the two smaller molecules, if heat is gained by the reaction and the surroundings turn colder, it would be an endothermic chemical reaction and should be described as an endothermic condensation reaction if that is the case as opposed to an endothermic reaction that does not have a condensation element. They are entirely different individual reactions so should not be confused as the same, even if there happens to be one reactant molecule that is the same in each.

Furthermore, an endothermic or net negative heat of solution is not the same as an endothermic chemical reaction because new molecules are not formed with the former. To illustrate this point let us take the example of ammonium nitrate, which can be involved with exothermic chemical reactions and with both kinds of endothermic reactions also, including one that liberates free water. When mixed with water by itself ammonium nitrate forms an intense net negative or endothermic heat of solution and the combination turns cold, but new molecules are not formed nor is water involved chemically other than as a solvent. On the other hand, when the same ammonium nitrate is mixed with barium hydroxide octahydrate the mixture again turns cold, but this time some new molecules are formed, 10 moles of free water, two moles of ammonia and the barium atom forms barium nitrate. The first example is an endothermic heat of solution reaction and the second is an endothermic chemical reaction with two new additional moles of water formed, but in this case as opposed to the usual condensation reaction, the largest single molecular weight is on the reactant side, not the result side of the chemical equation.

Only the results of any such reactions, if novel, are patentable, and the basic chemical reaction that played a role in their synthesis is not. Patents for such chemical reactions themselves are not being applied for in this disclosure, only the novel results. Since D-ribose has been synthesized since Emil Fischer's time to produce other molecules such as monoacetone-d-ribose, riboflavin, etc., attention being paid to the actual chemistry of the free molecule itself was not an issue for the FDA but only the synthesized vitamin, nucleoside, nucleotide, etc. In 1998 the synthesized form of crystalline free D-ribose was begun to be offered to consumers. The Dietary Supplement Health and Education Act (DSHEA) after Oct. 15, 1994 required that this product be classified as a new dietary ingredient and on Oct. 28, 1997 it was done so for D-ribose crystals, but done with inaccuracy. Nevertheless, for the first time the way it affects large populations could be ascertained and also what is the actual chemistry of the bulk-produced product now that it is intended for direct consumption by the populace, presumably with proper labeling and precautions, some of which will be addressed herein.

This invention is designed to overcome the deficiencies of previous applications and inventions by disclosing the exact molecule that synthesized D-ribose crystals become when coming in contact with water, including inside a living being and special derivatives of that molecule and its precursor.

### BRIEF SUMMARY OF THE INVENTION

This disclosure seeks to answer questions about a substance that has been biosynthesized for mass distribution for human consumption with little or no research having been done on exactly what it is in its synthesized artificial free-state. A simple way to start establishing what it is actually is by comparing its stated melting point to other free sugars that are closely related to it by name or use. For example, its epimer D-xylose melts at 148-153° C. while xylose's isomer, L-xylose, is at 150-152° C. D-glucose is at 153-156° C. while L-glucose is also at 153-156° C. The endothermic sugar xylitol has a melting point of 92-96° C. With respect to D-ribose its melting point is reported to range all the way from 80-92° C. (although we have found that it melts and starts caramelizing at the low, when sustained for as long as one half hour with laboratory sized samples, temperature of ±65° C.) whereas its isomer L-ribose is in a narrow range of 82-83° C.

If by not existing naturally in the free-state in nature, yet its radical being one of the most, if not the most, vital molecule or at least chemical radical in life, and by requiring considerable energy to be synthesized to its environmentally unnatural free-state of crystalline D-ribose, can it remain in the free-state when encountering the body, or is it too unstable to do so? If it is too unstable it should form a new molecule when put inside the body. Accordingly it was decided to put powdered crystalline D-ribose into the mouth and see what happens. If something does happen in vivo, then determine what could be done in vitro to duplicate it. When this is done, the immediate reaction is endothermic in that there is a feeling of coolness on the tongue. This same thing happens when xylitol powdered crystals are put on the tongue which is commonly attributed to a net endothermic heat of solution, however, the xylitol does not change into another molecule, remaining the same color but only becoming damp.

On the other hand, D-ribose does change into a different appearing substance, a light brown, non-translucent, no longer crystalline substance. Since the result becomes cold in the mouth like xylitol, the question arises, what kind of reaction is this? If a chemical reaction is taking place it has to be on a molecular basis. Since D-ribose has a molecular weight rounded off to 150 (listed at 150.13) and water 18 (listed at 18.015), if 12 grams of water were mixed with 100 grams of the powdered sugar and it were like xylitol, it would simply appear as damp crystals, however D-ribose becomes a completely different substance. This substance is a very viscous fluid or a soft semisolid, light brown in color, non-translucent, and quite sweet, and since the water cannot be driven off by heat at ambient pressure, it has a permanent molecular weight of 168 rounded off. It is a fluid at 100° F., whereas the crystals melt at a much higher temperature, closer to 150° F. It has a Maillard reaction capability above the level of D-xylose. When allowed to settle at room temperature it becomes a uniform soft light brown tacky solid, still non-translucent, that is highly soluble in water but now does not have a net negative or endothermic heat of solution and is quite stable unless heated.

Since life is based on water being ubiquitously present, this novel, newly discovered molecule lends credence to the special role that D-ribose has in life and the fact that it cannot exist in the free-state inside living beings, so cannot be produced in the free-state in living beings. This is why glucose which does exist in the free state cannot be converted enzymatically or non-enzymatically to free ribose but must first become a radical itself, forming glucose-6-phosphate, before the pentose radical can be formed into ribose-5-phosphate in the pentose phosphate pathway. Of course, once in the body the new monohydrate molecule of this disclosure can form ribose-5-phosphate directly, shortcutting the tedious pentose phosphate pathway.

This new molecule exists at low temperatures such as room temperature and at body temperature also, but as it is heated further it becomes increasingly translucent and decreasingly viscous as it caramelizes. This brings up a comparison between D-ribose crystals and the molecule they become when placed in the body (in vivo). When heated in vitro at temperatures higher than body and/or room temperature there is a difference between the monohydrate and the powder. Whereas the monohydrate is a non-translucent fluid at 100° F., the powdered crystals remain crystalline at that temperature. As the crystals approach their melting point they also caramelize becoming a translucent brown fluid. At this temperature, ±65° C., both the monohydrate and the crystals are in a caramel-appearing state that when returned to room temperature is a translucent soft solid in both cases. Kept at high temperature it is not of ongoing physiologic importance since the body cannot operate at such high temperatures, but the cooled caramelized product can be consumed in either case with a possible downside. Caramelizing D-ribose above 100° C. in the presence of food may enable the resulting Mail lard reactions to form acrylamide at a much lower temperature than is the case for a hexose sugar which ribose-caramel then may have mutagenic potential at lower temperatures than glucose when used in cooking. Because of ribose's early Maillard reaction, meat can be browned at lower cooking temperatures, before dextrose and sucrose can be available for the Maillard reaction. This is mostly attributed to the ribose compounds such as adenosine triphosphate or ATP in meat. ATP and other nucleotides and nucleosides are also in plant foods including potatoes, but higher temperatures are needed here for culinary purposes, which enable many free reducing sugars to form acrylamide.

Thus the monohydrate molecule of this disclosure is for ongoing physiologic use at body temperature or below unless it is being used as a precursor molecule for other chemical reactions. It is to be used either inside the body or on the skin at body temperature or below, unless submitted for approval by the FDA for other products after caramelizing, such as for cosmetics or drugs. The fact that it starts to caramelize readily at ±65° C. indicates how reactive it is and thereby dangerous for animal consumption as a food additive if cooked at high temperatures above 100° C. with animal and even plant food. At elevated temperatures above 100° C. while being used to flavor meat by the burnt sugar taste, it may form mutagens such as acrylamide. Just because ribose compounds are already in meat and plant foods and form Maillard reactions is no reason to add synthetic crystallized ribose to the problem if mutagens are potentially increased thereby. Because of its special reactive nature it tends to start caramelizing at temperatures below 85° C., but the process continues more rapidly above that point. By that process it becomes a different molecule or many different molecules due to that poorly understood chemistry that borders on the same lack of understanding of the Maillard reaction. Under the conditions disclosed herein, D-ribose monohydrate is a novel new molecule with a therapeutic use with respect to the living body at body temperatures.

It can be mixed with certain foods safely at body temperature as an alternative sugar for diabetics and those who are dieting and don't want hyperglycemic spikes. Nevertheless, warnings need to accompany labeling not to cook with ribose in order to further flavor animal-derived foods above 100° C. One only has to consult the FDA search engine to learn the potential danger of cooking with synthetic powdered D-ribose. The molecules including also their caramels of this disclosure should not be used for cooking at high temperatures with our present state of knowledge because of the following information from the FDA:

Food Advisory Committee

Feb. 24-25, 2003 Meeting

Acrylamide

FROM Transcript of Proceedings Feb. 24, 2003

“Are there other carbonyl sources that can form acrylamide? Some recent work speculated that the formation of acrylamide from asparagine, the structured degradation reaction—structured degradation reaction is implicitly explained, actually a di-carbonyl such as, in this case, glyoxal reacting with the amino acid causes the reaction to proceed. We also showed that glyceraldehyde, 2-deoxyglucose and ribose are also efficient at forming acrylamide in food systems”.

Mixing powders is always an uneven and tedious process, while mixing a powder with a liquid is quick and easily made uniform. To do so with powdered D-ribose with any speed it must be heated to around 180° F. and then the powder such as whey, soy, starch or even another sugar mixed in. This leads to the danger of acrylamide formation if temperatures above 100° C. (212° F.) are encountered. On the other hand, the monohydrate of this disclosure need be only heated to body temperature or around 1000 F, and it will turn into a fluid that aids mixing, especially with chocolate which melts at low temperatures, without any danger of new acrylamide formation as long as it is consumed without extreme heating further on.

Even so, what precursor actually causes the most acrylamide in cooking and baking is still not decided. Of three likely precursors, ribose is the most Maillard reactive, although its phosphorylated compounds do not appear to form acrylamide; glucose along with fructose is present in much larger amounts, and it is possible that acrolein formation from glycerol and subsequent oxidation and amidation into acrylamide may contribute, however, acrylamide does not form in food prepared at body temperatures. According to Proctor & Gamble scientists, Zyzak, et al. in the 2003 article, “Acrylamide Formation in Heated Food”, 2-carbon atom glyoxal is the most reactive, while 5-carbon atom ribose is more reactive than 6-carbon atom glucose, all when reacting with asparagine to form acrylamide. While the physiologic use of the monohydrate molecule of this disclosure is at temperatures no higher than body, its reactions at higher temperatures are pertinent to its understanding and restraint of use, especially with any tendency to form toxic compounds at high temperatures in cooking.

The features of the present invention which are believed to be novel are set forth with particularity. The present invention, both as to its organization and the manner of operation, together with the further objects and advantages thereof, may be best understood by reference to the following exemplary and non-limiting detailed description of the invention, wherein;

### DETAILED DESCRIPTION OF THE INVENTION NO DRAWINGS

The preferred embodiment is to mix one mole (MW 150) only, or direct increases or decreases thereof only, of D-ribose anhydrous crystals with one mole (MW 18) only, or direct increases or decreases thereof only, of water at room temperature and obtain a new molecule that now has a molecular weight of 168 and is now uniformly amorphous, forming a uniform soft solid that is light brown, sweet, non-translucent, becomes fluid at as low as ±100° F., and does not resemble its crystalline precursor. No part of the water can be driven off by evaporation at room temperature at sea level, and even with heat applied at such ambient pressure it loses mass mostly by chemical rearrangement sometimes referred to as caramelizing or oxidation that forms by prolonged heat well above body temperature so becoming then a different molecule or molecules than is the primary one of this disclosure. The primary molecule of this disclosure would be presumed to be D-ribose monohydrate (C sub 5H sub 10 O sub 5 monohydrate or C₅H₁₀O₅.H₂O). This substance is highly soluble in water, whereas crystalline D-ribose's water solubility is not measurable because the monohydrate is formed first, and it is what goes into solution.

Thus it would be logical in hindsight since it was not anticipated, that such a highly reactive molecule as crystalline D-ribose could not exist free but only as a radical in nature. Therefore, it would form a compound of some sort when entering in vivo the chemical machine that is the living body, and this chemical could also be synthesized in vitro which this disclosure bears out. Actually free-state D-ribose itself is quite unstable if exposed to chemicals in the atmosphere or on earth such as water, protein and even heat at comparatively low temperatures. Whereas xylitol (which will melt but not caramelize) and other free sugars exposed to heat at the boiling point of water (212° F.) can withstand such temperatures and remain in their free state, crystalline D-ribose will become a light brown or amber fluid fairly rapidly at temperatures considerably below this indicating one reason why it does not exist in the free-state in nature. Since it caramelizes or oxidizes at fairly low temperatures it is far more unstable than glucose. That is why crystalline D-ribose does not behave as other sugars, and this extends to reacting with water at room temperature to form its simplest compound, its newly discovered monohydrate, the primary subject of this disclosure.

Since this monohydrate can be formed in various ways when D-ribose compounds such as ribonucleic acid or RNA are being subjected to being a precursor for the biosynthesis of free D-ribose, wherever its monohydrate is formed is subject matter for this disclosure, so D-ribose monohydrate can be harvested as soon as it appears in any process. Furthermore, since D-ribose monohydrate is very soluble in water, it can be marketed as a concentrated water solution.

D-glucose forms a monohydrate, and glucose is the precursor of the ribose radical in the pentose phosphate pathway. Thus, a comparison of the two monohydrates is in order. First let us compare the caramelizing properties of the two crystalline sugars. At low temperatures glucose remains a powder below its melting point at ±150° C. and becomes caramelized at about 160° C. On the other hand over time of as little as a half hour, ribose forms an irreversible caramel-appearing, sticky semisolid at as low a temperature as 65° C., not far above body temperature compared to glucose. At this point it no longer exhibits an endothermic capability when the substance is reduced to room temperature and subjected to water. Its molecular weight remains at 150.

D-Glucose monohydrate is commonly called dextrose monohydrate, and it comes from the enzymatic hydrolysis of starch, usually corn starch, and when under controlled cooling, dextrose monohydrate crystals are precipitated. Whereas dextrose monohydrate and dextrose itself can coexist in the free-state, and dextrose monohydrate can be converted to dextrose simply by heating, D-ribose monohydrate cannot. Although at room temperature, ribose monohydrate is a non-translucent, soft, light brown substance, on becoming a fluid above body temperature it becomes translucent which does not diminish as it becomes less viscous gradually at higher temperatures. When heated at 100° C. (212° F.) over time it will gradually lose mass, but not its translucent appearance only becoming a darker amber. After being heated at 100° C. it does not lose its sweetness but has a more burnt taste on being cooled, a property it shares with caramelized powdered D-ribose and also the most common molecule used for commercial caramelizing, sucrose, but all only when cooked long enough or at higher temperatures than their respective melting points. After 90 grams of this amorphous soft solid being heated arbitrarily at the boiling point of water, well above its initial caramelizing point of ±65° C., its mass over time compared to that of the precursor powder changes as follows:

| Mass monohydrate (grams) | Mass powder (grams) | Time (minutes) |
|---|---|---|
| 90 | 79.2 | 0 |
| 89 | 79.2 | 30 |
| 88 | 79.2 | 60 |
| 86 | 79.2 | 90 |
| 86 | 79.2 | 150 |
| 84 | 79.2 | 210 |
| 84 | 79.2 | 270 |
| 84 | 79.2 | 330 |

The loss of mass appears to be confusing. It could be due to the rearrangement of the entire molecule with loss of carbonyl-condensed water being driven off, or loss of some of the water of hydration or both, but it points out the fact that at room and body temperatures, even though the caramels are similar, the monohydrate is still different in kind from the powder as can be realized by comparing it to dextrose and its monohydrate.

With respect to dextrose monohydrate there involves a total loss of water of hydration. The non-total ribose monohydrate weight loss therefore could be due to condensation with the release of water molecules that are not part of hydration but of chemical destruction of the radical itself, but this does not happen to crystallized D-ribose heated under the same conditions. If it were due only to evaporation of the water that formed the hydrate at room temperature, ribose monohydrate would have lost mass down to 79.2 grams. A complete loss of its bound water is what happens to dextrose monohydrate when heated only to 80° C. for two hours. This is well below its caramelizing point of ±160° C., but ribose monohydrate starts to oxidize at a much lower temperature.

Now the ribose monohydrate when cooled after being so heated, returns to a soft solid form but of a darker amber color, keeping this color and its translucency at room temperature and appears very much like crystallized D-ribose subjected to the same temperature and then cooled. They both have the burnt sugar taste. Of course, ultimately at high enough temperatures both the ribose monohydrate caramel and the ribose powder caramel will destructively decompose and lose mass to below 79.2 grams due to volatile components of the decomposition products. The ribose caramels, either one, when cooled have uses in topical skin products including cosmetics, but neither are the same as they were before the caramelizing occurred. Of course before caramelizing ribose monohydrate has use both in aqueous and non-aqueous skin formulations but powdered D-ribose only in non-aqueous products or it will become the monohydrate.

Since dextrose monohydrate starts decomposing (it has already lost its water of hydration) at crystalline dextrose's melting temperature (±150° C.), followed by caramelizing at about 10 degrees higher, if ribose followed the same general chemistry of free sugars, one would expect D-ribose monohydrate to lose its water of hydration and start to decompose at crystalline D-ribose's melting temperature, reported in patent number U.S. Pat. No. 4,602,086 as ±85° C. (but discovered to be ±65° C. as a result of measurements for this disclosure) which is still low when compared to glucose. When not subjected to limitations of temperature, caramelizing should continue until total destruction of the conglomerate being oxidized is reached—carbon, carbon dioxide and water being the major end products. Sucrose and glucose have rather sharp caramelizing and melting point temperatures but ribose with respect to any stated melting point is at a far lower temperature. Thus with respect to D-ribose, the full mutagenic potential of both Maillard reaction and caramelizing remains to be discovered, but it would make sense not to cook food with either crystalline ribose or its monohydrate above 100° C. with our present knowledge, however, the ribose caramel below 100° C. has many uses.

The fact that the pentose polyol, xylitol, does not caramelize, but D-ribose does at a low temperature makes it possible to make a partial caramel combining the two at a temperature below 100° C. with contributions from both having the physiological benefits of ribose and the tooth-decay prevention of xylitol, including without hyperglycemia. This would extend to the polyol, erythritol, but it melts at a much higher temperature and is not as sweet. Other ingredients such as chocolate can also be mixed in. To show how the endothermic nature of xylitol is different from that of ribose, if 50% D-ribose monohydrate is mixed with 50% xylitol and heated to 100° C. then allowed to cool to room temperature and the same thing done with 100% D-ribose monohydrate, making a caramel, the xylitol-ribose mixture will still have an endothermic feeling on the tongue but the 100% ribose will not.

The taste of caramelized ribose is different from the taste of ribose powder becoming ribose monohydrate in the mouth, but when brought to the caramel state both kinds of ribose taste virtually the same. This pleasant strong taste carries over to mixing ribose with xylitol to the extent that even as low as 10-20% caramelized ribose mixed with 80-90% xylitol will bear the light brown color of the ribose caramel and the taste of the caramel to a large extent with the temperatures of production-mixing capable of being kept below 100° C. Since a sugar undergoing caramelizing and kept at the same temperature is constantly changing in structure over time, the time of heating becomes an important factor with respect to the taste and texture of the “caramelized” xylitol. Since both ribose and xylitol melt below 100° C., they make a unique caramelized compound that is readily made by simply mixing ribose and xylitol powders together and heat at no more than 100° C. until a clear brown translucent liquid combination appears and then is allowed to cool. Since xylitol has a tendency to crystallize in place, the more thorough the mixing, the more uniformity, especially when ribose is less than 50%. The presence of some water makes xylitol's crystallization more rapid, and when both precursors are wetted, premixing before heating is cost effective. This would be especially useful when used to coat and flavor chewing gum.

Therefore, we have a pentose sugar, ribose, that is unstable in its synthetic crystallized form at room temperature, but becomes stable when a molecule of water is combined with it. Then if either the synthesized crystalline powder or its monohydrate is heated they become caramelized at a far lower temperature than the boiling point of water and then become virtually the same with respect to most physical properties and are stable. As long as they are not over-heated the caramels can be combined with other kinds of food or the caramels can be consumed by themselves. On the other hand, if cooking is not done, the monohydrate remains the embodiment of choice when subjected to ingestion by the body up to body temperature. When caramelized it takes the bland sweet taste of xylitol and changes it into a delicious caramel taste. Whereas no one would eat either crystallized ribose or crystallized xylitol by the spoonful, they will this tasty combination. It can be packaged as a bulk powder or as a confection. Care must be taken not to heat it with amino acids or food containing protein above 100° C. until the potential acrylamide danger at high temperatures is resolved.

To sum up some of the differences between the precursor powder and its resultant monohydrate, when synthesized D-ribose crystalline powder exposed to heat forms the reaction that appears like oxidation, starting out as the crystals but then it forms a translucent amber fluid at as low as ±65° C. (150° F.). It appears to look much like D-ribose monohydrate subjected to the same temperature, but the powder does not have any water of hydration attached so remains at the same lower molecular weight throughout the same heating cycle up to at least 100° C. as shown above. The two appear to be very close in texture and taste when cooled from this temperature, but some water remains in the bonds of the monohydrate. Therefore, even though appearing the same, the ribose monohydrate complex and the crystalline version have different molecular weights at least in part. As indicated above, the monohydrate complex now has an average molecular weight of closer to 160 while the other remains at 150. The actual chemical formulas of both partially oxidized products obviously will vary somewhat and are not defined in this disclosure with respect to specific molecular structure. They are both different from their original structures. Both the crystalline powder and the amorphous monohydrate have substantially different properties at room and body temperatures, but the differences diminish substantially with caramelizing.

Although ribose monohydrate cannot be completely desiccated by subjecting it to heat at 80° C. (175° F.) for 2 hours, dextrose monohydrate can. Desiccating dextrose monohydrate occurs simply by spreading its crystals onto a cooking sheet to a maximum depth of about 1 cm, then placing the sheet into a preheated oven set at that temperature. After heating then cooled, the resulting caked lumps now with 91% of the original weight may be broken up in a mortar and pestle or using an electric coffee grinder. Since D-ribose monohydrate forms spontaneously at room temperature and dextrose monohydrate only with controlled cooling from a much higher temperature, the degree to which the water molecule is attached to ribose monohydrate is much stronger than its attachment to dextrose monohydrate. Obviously this higher degree of reactivity is why glucose makes ribose in the pentose phosphate pathway and not the reverse in the scheme of life.

It becomes apparent that the synthesis of D-ribose monohydrate wherever it occurs and from whatever process being used results in a new molecule for ongoing physiologic use or being made into its caramel, both novel to the understanding of those skilled in the art. Since at this point the monohydrate can be harvested as a concentrated aqueous solution of it, if xylitol is to be mixed with it, xylitol can also be harvested before crystallization. Then by mixing the two liquids together and subjecting them to heat, the xylitol “caramel” can be produced without the expense of crystallizing either sugar. The same can be done with erythritol, hexose polyols including sorbitol, disaccharide polyols including maltitol, and even free sugars for natural caramel flavoring when high temperatures are contraindicated so as to avoid acrylamide and other mutagenic possibilities from forming via Maillard reactions.

While we have discussed this disclosure principally as it applies to human beings, it also applies to other animals and products designed for them.

While particular variations of the present invention have been described, it will be obvious to those skilled in the art that changes and modifications may be made without departing from my invention in its broader aspects of synthesizing the novel molecule, D-ribose monohydrate and utilizing both its and its precursor's novel caramels.

What is claimed:

1. A new molecule resulting from mixing one mole only, or direct increases or decreases thereof only, of water with one mole only, or direct increases or decreases thereof only, of crystallized, powdered D-ribose at room temperature.

2. The formation of the new molecule as in claim 1 inside the body thereby at body temperature.

3. The new molecule of claim 1 being D-ribose monohydrate (C sub 5H sub 10 O 12 sub 5 monohydrate or C₅H₁₀O₅.H₂O).

4. The new molecule of claim 1 being formed by an endothermic chemical reaction.

5. The new molecule of claim 1 being an amorphous light brown, non-translucent, tacky, semisolid, sweet substance that melts at ±100° F.

6. The new molecule of claim 1 being able to react with the carboxylic acid moiety of amino acids.

7. The new molecule of claim 1 having the ability to caramelize.

8. The new molecule of claim 1 having a high degree of water solubility.

9. The new molecule of claim 1 having a stronger ability to retain its monohydrate than does dextrose monohydrate.

10. The new molecule of claim 1 being harvested in fermentation processes, including with Bacillus subtilis, before crystallization means are employed.

11. D-ribose being mixed with xylitol and heated to D-ribose's caramel status.

12. D-ribose being mixed with erythritol and heated to D-ribose's caramel status.

13. D-ribose monohydrate being mixed with xylitol and heated to D-ribose monohydrate's caramel status.

14. D-ribose monohydrate being mixed with erythritol and heated to D-ribose monohydrate's caramel status.

15. D-ribose monohydrate being mixed with a concentrated aqueous solution of xylitol and then heated to D-ribose monohydrate's caramel status.

16. D-ribose monohydrate being mixed with a concentrated aqueous solution of erythritol and then heated to D-ribose monohydrate's caramel status.

17. D-ribose being mixed with hexose-containing sugars then heated to D-ribose's caramel status.

18. D-ribose monohydrate being mixed with hexose-containing sugars then heated to D-ribose monohydrate's caramel status.
